(19) United States
(12) Patent Application Publication
(10) Pub. No.: US 2009/0043245 A1
(43) Pub. Date: Feb. 12, 2009

(54) DEVICE FOR ADMINISTERING AN INJECTION AND METHOD OF USING SAME

(76) Inventor: Li Nguyen, Westminster, CA (US)

Correspondence Address: CHRISTIE, PARKER & HALE, LLP, PO BOX 7068, PASADENA, CA, 91109-7068, US

(21) Appl. No.: 11/721,673

(22) PCT Filed: Dec. 22, 2005
(86) PCT No.: PCT/US2005/047082

§ 371 Date: Mar. 31, 2008

Related U.S. Application Data
(60) Provisional application No. 60/638,829, filed on Dec. 22, 2004.

(51) Int. Cl. A61N 1/30

(52) U.S. Cl. 604/21

(57) ABSTRACT

Device for administering an injection is generally discussed herein with particular discussions extended to devices for stimulating a cavity below the skin and administering a dosage in the stimulated cavity and method of using the same. The dosage can be medication for health and/or cosmetic treatments. The device includes a housing and a needle mounted thereto. A vacuum source is in communication with the housing for creating a vacuum inside the housing while a power source is in communication with the needle for supplying current to the needle. The combination current and vacuum stimulates a cavity for injection. A vacuum source may also be used to advance a piston to discharge medicament from a needle mounted on a syringe.

Device for administering an injection is generally discussed herein with particular discussions extended to devices for stimulating a cavity below the skin and administering a dosage in the stimulated cavity and method of using the same. The dosage can be medication for health and/or cosmetic treatments.

### BACKGROUND

There are a number of means for delivering medications for treatment of diseases and illnesses including orally and via a syringe. Historically when using a syringe to deliver medications, a subject will generally be poked by a needle, either in the arm, leg, or buttock, and the medications delivered to the injection site through the lumen in the needle. The injection can vary from one caregiver to another as the location and depth of an injection can vary from one caregiver to another, which, for the most part, is not critical.

More recently, syringes are used to deliver medications for treating wrinkles, i.e., for cosmetic reasons. In these instances, a caregiver will randomly inject wrinkle areas of the face at varying depths to deliver neurotoxin, such as BOTOX®, or injectable fillers, such as ZYDERM®, ZYPLAST®, RESTYLANE®, RADIANCE®, and ARTFILL™, just to name a few. As these medications are injected to wrinkle areas at random depths, their effectiveness and efficiency are less than optimal.

Accordingly, there is a need for a device that ensures a more consistent delivery of medications and method of using same. Such a device not only can be used for treating wrinkles but can also be used for non-cosmetic treatments, such as for delivering anesthesia and slow release birth control medications.

### SUMMARY

The present invention may be implemented by providing an injection apparatus comprising a needle assembly comprising a needle mounted to a housing; a vacuum source connected to the housing for creating a vacuum inside the housing; and a power supply directly or indirectly connected to the needle for supplying a current to the needle.

The present invention may also be practiced using a method for injecting a subject comprising (a) placing an injection apparatus on a skin of the subject; the injection apparatus comprising: (1) a needle assembly comprising a needle mounted to a housing; (2) a vacuum source connected to the housing; (3) a power supply directly or indirectly connected to the needle for supplying a current to the needle; (b) penetrating the skin of the subject with the needle; and (c) delivering an injection to the subject.

In yet another aspect of the present invention, there is provided a method for injecting a subject comprising penetrating a skin with a needle; stimulating a cavity below the skin using an electric stimulator and a vacuum source; and delivering medication to the cavity.

In still yet another aspect of the present invention, there is provided a method for injecting a subject comprising: penetrating a skin with a needle; stimulating a fascia layer below the skin with an electric stimulator; and delivering a medicament adjacent the stimulated fascia.

In yet another aspect of the present invention, there is provided an injection apparatus comprising an injection module housing defining an interior cavity comprising a nozzle for connecting to a vacuum source, a first receiving end for receiving a syringe, and a second receiving end for accommodating an end object for sealing the second receiving end, wherein the nozzle is configured to communicate with a vacuum source to place the interior cavity under a vacuum.

### BRIEF DESCRIPTION OF THE DRAWINGS

These and other features and advantages of the present invention will become appreciated as the same become better understood with reference to the specification, claims and appended drawings wherein:

FIG. 1 is an exemplary schematic diagram of a skin tissue;

FIG. 2 is a schematic diagram of an injection assembly provided in accordance with aspects of the present invention;

FIG. 3 is a semi-schematic cross-sectional side view of an exemplary needle assembly provided in accordance with aspects of the present invention;

FIG. 4 is a schematic diagram of an alternative injection assembly provided in accordance with aspects of the present invention;

FIG. 5 is a schematic diagram of the injection assembly of FIG. 2 used on a subject;

FIG. 6 is a semi-schematic diagram of the skin tissue following an injection using an injection device provided in accordance with aspects of the present invention

FIG. 7 is a semi-schematic diagram of an alternative injection assembly provided in accordance with aspects of the present invention;

FIG. 8 is a semi-schematic diagram of the injection assembly of FIG. 7 with an accordion seal attached to the elongated shell;

FIG. 9 is a semi-schematic diagram of the injection assembly of FIG. 7 with a leveling plate; and

FIG. 10 is a semi-schematic diagram of an alternative injection assembly which uses a vacuum source to advance a plunger for delivering medications from a syringe.

### DETAILED DESCRIPTION

The detailed description set forth below in connection with the appended drawings is intended as a description of the presently preferred injection devices, which are exemplary embodiments provided in accordance with aspects of the present invention, and is not intended to represent the only forms in which the present invention may be constructed or utilized. The description sets forth the features and the steps for constructing and using the injection devices of the present invention in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

This application is related to Ser. No. 10/932,751, filed Sep. 2, 2004, entitled INTEGRATED NEUROTOXIN INJECTION SENSING AND CONTROL DEVICE, the contents of which are expressly incorporated herein by reference as if set forth in full.

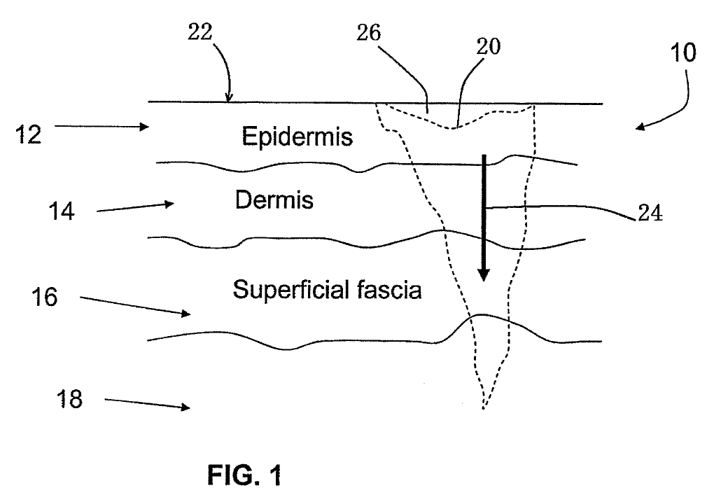

Referring now to FIG. 1, there is shown an exemplary schematic cross-sectional view of a skin tissue of a human body, which is generally designated 10. As is well known in the medical field, the skin tissue 10 comprises a skin layer, which are the epidermis 12 and dermis 14 layers, a superficial fascia layer 16, a deep fascia layer, and a muscle layer, shown collectively as 18. In the facial area however, there is no deep fascia layer. Instead, facial muscles are embedded in the superficial fascia layer 16. The superficial fascia layer 16 in the facial area, herein the fascia 16, thus comprises a loose network of connective tissue bundles, collagen, and elastin, which blend with the dermis.

Wrinkles have been described as aging, sun-damaged skin, the loss of elastin and/or collagen, etc. Whether or not due to one or more of these reasons, from the schematic shown, a wrinkle 20 on the skin outer surface 22 is simply an outward reflection of the movement 24 of muscles underlying the skin along an axis perpendicular to the skin, i.e., the Z axis. Thus, as muscles are embedded in the superficial fascia layer 16 in the facial region, wrinkles in the facial region is movement of the fascia layer 16 along the Z axis. A void space 26 formed from the original skin contour 22 and the wrinkled skin 20 are thus both formed due to movement of the fascia layer 16.

In one aspect of the present invention, wrinkle treatment involving recovering at least part of the void space 26 is provided by injecting medications, which can be a filler or a neurotoxin, below the skin 22 to either (1) fill-out the skin to create a more even line with the top skin contour 22 or (2) paralyze the muscles that cause the skin to wrinkle 20 so that they relax and not contract along the Z axis. More preferably, medications are injected in between the dermis layer 14 and the fascia 16 to treat wrinkles. Most preferably, medications are injected in a cavity (not shown) formed between the dermis layer and the fascia using a device of the present invention for consistent and prolonged wrinkle treatment, as further discussed below. Other medications not presently approved for wrinkle treatment but in the future are approved may also be used with the devices of the present embodiment provided they are useable with a syringe.

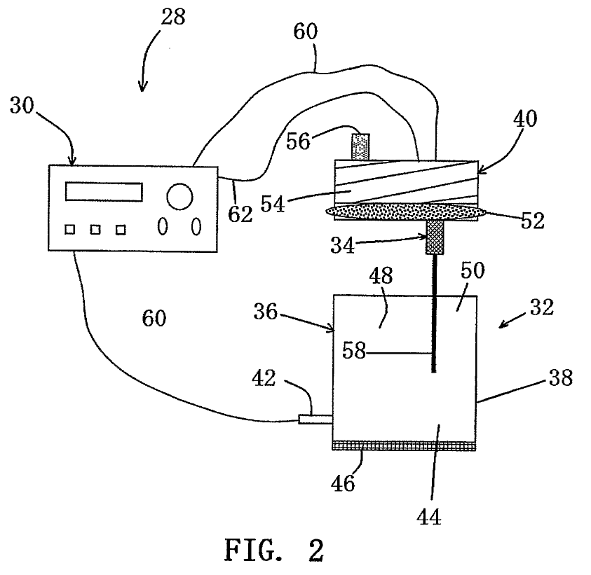

Referring now to FIG. 2, an exemplary injection assembly for delivering an injection in accordance with aspects of the present invention is shown, which is generally designated 28. In one embodiment, the injection assembly 28 comprises a master controller 30 and an injection module 32 comprising a needle assembly 34 mounted to a housing 36. In general, the master controller 30 is configured to supply an electrical stimulation to the facial tissue via the needle assembly 34, a vacuum to the housing 36 to create a pulling force on the skin 22, and pressurized gas to the needle assembly 34 to inject medications, as further discussed below. The injection module 32 is configured to penetrate the skin, transfer low voltage to provide stimulation to the facial tissue, and deliver medications.

In one exemplary embodiment, the master controller 30 comprises a vacuum pump (not shown), pressurized gas supply (not shown), a power supply device (not shown), and electronics for regulating the vacuum pump, gas flow, and either voltage and/or amperage supplied by the power supply device. The injection module 32 comprises a wall enclosure 38, a cap 40, and the needle assembly 34, as previously discussed. The injection module 32 also comprises a nozzle 42, a first open end 44 comprising a pliable seat 46, and a second open end 48 comprising a mating connector 50, which in one exemplary embodiment is a corresponding threaded end for receiving the cap 40. The nozzle 42 is preferably a hose barb connector for connecting to an air hose and may include a rocker pinch valve as disclosed in U.S. Pat. No. 6,340,096. The pliable seat 46 is preferably a foam-based gasket but may be a rubber-base gasket removably adhered to the wall enclosure end with adhesive. In one exemplary embodiment, the pliable seat 46 is disposable and comprises pressure sensitive adhesive for providing a vacuum tight seal with the face, as further discussed below. The pliable seat 46 comprises an opening and a configuration that matches the configuration of the wall enclosure 38, which in one exemplary embodiment is a cylindrical wall enclosure having two open ends 44, 48.

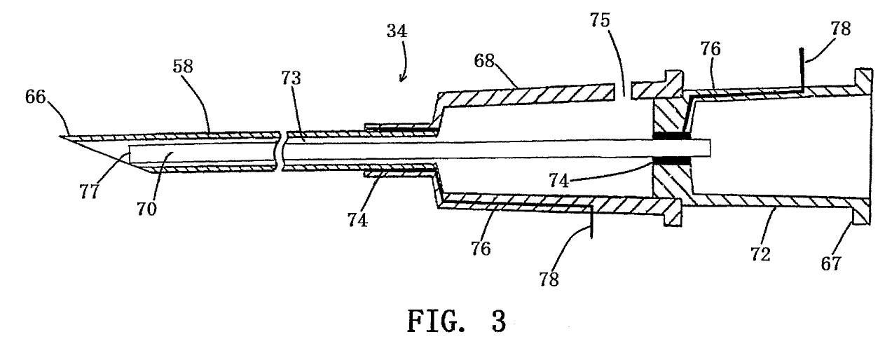

The cap 40 comprises an O-ring 52 seated in a groove, a mating connector 54, a vacuum breaker 56, a receiver (not shown) for receiving the needle assembly, a pneumatic connector (not shown) for connecting to the pressure source, and a terminal connector (not shown) for connecting to the power supply. In one exemplary embodiment, the vacuum breaker 56 is configured for manual opening by turning a valve or a piston to open the housing to atmosphere to enable removable of the housing. As is readily apparent to a person of ordinary skill in the art, any of the various components on the wall enclosure 38 and the cap 40 may be located elsewhere on the wall enclosure and the cap. For example, the vacuum breaker 56 may be located on the wall enclosure 38 instead of on the cap 40 and the nozzle 42 may be located on the cap 40 instead of the wall enclosure 38. Furthermore, instead of using threads to couple the cap 40 and the wall enclosure 38 together, in an alternative embodiment, detents or straps may be used. Preferably however, threads are used to enable adjustable engagement of the cap 40 to the wall enclosure 38 to thereby adjust the length of the needle 58 that extends distally of the edge of the pliable seat 46. In one exemplary embodiment, a reservoir for storing medications is located on the cap and the needle assembly, particularly the dispensing hub 72 (FIG. 3), is in communication with the reservoir.

The master controller 30 is placed in communication with the injection housing 36 by connecting the nozzle 42 to the vacuum pump using a hose 60, connecting the pressurized gas to the pneumatic connector on the cap 40 using a hose 60, and connecting the terminal connector on the cap to the power supply device using a cable 62. In one exemplary embodiment, the power supply device and the vacuum pump are both located inside the controller 30 while the pressurized gas supply is external to the controller. However, the pressurized gas supply, which may be an air pump, may also be located inside the controller 30. Preferably, the vacuum source and the gas supply are both external of the controller. For example, the vacuum source can be part of a central vacuum source and the gas supply can be a pressurized gas tank, of either nitrogen or air. The controller 30 preferably controls current flow and pressurized gas to the needle assembly 34 as well as timing of the current flow and the gas flow for reasons further discussed below.

Referring now to FIG. 3, a needle assembly 34 provided in accordance with aspects of the present invention is shown. The needle assembly 34 resembles a catheter assembly and comprises a needle 58 having a sharpened needle tip 66 attached to a needle hub 68, and a tube 70 attached to a dispensing hub 72. In one exemplary embodiment, the dispensing hub 72 comprises an engagement end 67 configured to engage the receiver on the cap 40. The dispensing tube 70 and the needle 58 in the present embodiment are both made from a metallic material, preferably of stainless steel. In one exemplary embodiment, the needle hub 68 and the dispensing hub 72 are both co-molded with a metallic insert 74 comprising a metallic strip 76 comprising an exposed lead 78. The two leads 78 are configured to couple to a power supply device to impart an electric current to the facial tissue for stimulating a cavity, as further discussed below. The metallic inserts 74 may each comprise a cylindrical configuration or an open curved metallic section configured to contact with the needle.

In one exemplary embodiment, a gap or space 73 is provided in the annular space between the needle 58 and the dispensing tube 70. This gap 73 is in communication with an opening or vent port 75 incorporated in the needle hub 68. Thus, when the needle 58 is inserted into a skin tissue, the area of the skin tissue that surrounds the needle tip 66 is in fluid communication with the vent port 75, which is in communication with the atmosphere. As further discussed below, fluid to be dispensed by the needle assembly 34 is dispensed through the dispensing hub 72 and dispensing tube 70 and out of the end opening 77 of the tube. In an alternative embodiment, a plurality of vent ports 75 are incorporated in the needle hub 68. In yet another aspect of the present invention, the needle tip 66 of the needle 58 comprises a non-coring tip, which typically includes a bend in the shaft.

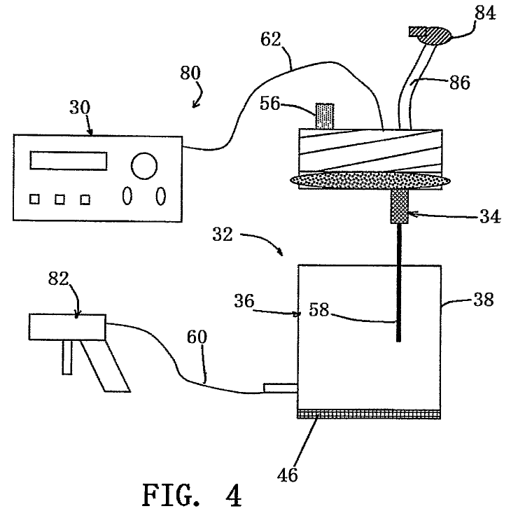

FIG. 4 is an alternative injection assembly 80 provided in accordance with aspects of the present invention. In the present embodiment, a hand vacuum pump 82 is incorporated for providing a vacuum and a hand activated valve 84 connected to a line 86 and in communication with the needle assembly 34 for regulating medication flow out of the dispensing tube 70. The valve 84, when activated to open, is opened on one side to the atmosphere.

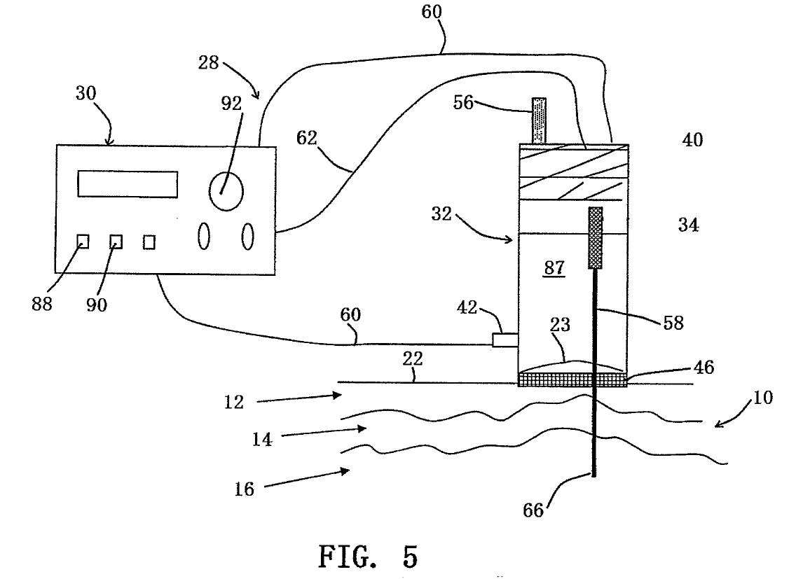

Referring now to FIG. 5, the injection assembly 28 is shown used on a patient. In an office setting, a subject or patient is first directed to lay down in a supine or semi-recumbent position in a chair and the face to be treated is positioned substantially horizontally. The injection module 32, with the needle 58 adjusted to extend about 0.4 cm to about 1.5 cm distally from the end of the pliable seat 46 and the various connectors and lines connected to the master controller 30, is then placed on the facial skin 22 of the patient. However, the length can vary depending on the treatment and location of injection. The injection module 32 should be placed directly over a wrinkle to be treated. Placement of the module 32 results in the needle 58 penetrating the skin at the wrinkled area to a depth set by the position of the needle tip 66 relative to the pliable seat 46. If the injection module 32 is connected to an external vacuum source, a vacuum is created inside the interior cavity 87 of the wall housing 36 without initiating the master controller 30 otherwise a vacuum power switch 88 on the master controller 30 is activated to initiate the vacuum pump for generating a vacuum in the interior cavity. A soft vacuum of about 7 to about 14 psia should be established inside the interior cavity. A vacuum pressure transducer may be incorporated to verify the vacuum inside the cavity. Preferably the vacuum is kept to about 9-13 psia. The skin 23 under the vacuum bulges outwardly into the interior cavity 87 of the housing 36, which is shown exaggerated for discussion purposes.

Once a sufficient vacuum is established, a current is sent to the needle assembly 34 by activating a power source switch 90. A current of about 1.5 mA to about 5 mA supplied to the needle is preferred with a current of about 2 mA to about 3 mA being more preferred. The higher the current, the more the fascia 16 will contract, as further discussed below. In one exemplary embodiment, the controller 30 has built-in electronics to regulate the amount of current output to the needle 58, which may be adjusted by turning a dial 92.

The current provided by the controller 30 to the terminal connector (not shown) located on the cap 40 and then to the needle 58 and dispensing tube 70 via the leads 78 on the needle hub 68 and dispensing hub 72 causes the muscles adjacent the needle and dispensing tube to contract. The contraction is caused by an electrical stimulation to an area located around the needle that is known as the neuromuscular junction. Current discharged in this region produces a muscular response. The contraction is caused by an electrical stimulation to an area located around the needle, and therefore first will stimulate the neuromuscular junction lying within the fascia that is adjacent to the needle body. Current discharged in this region produces a muscular contraction following the release of acetylcholine, which initiates an action potential and this then propagates through the rest of the muscles.

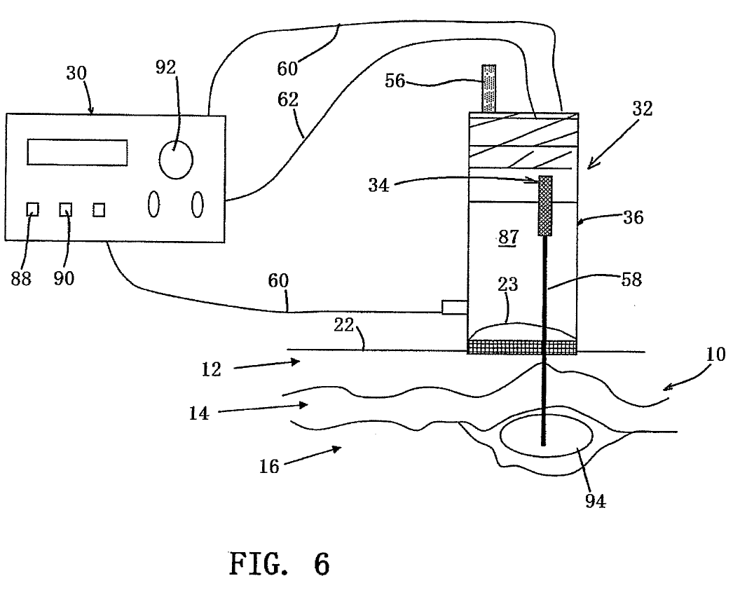

The muscles, which as previously discussed are embedded in the fascia, move away from the current source, i.e., the needle. Normally this causes the skin 22 to move with the fascia. However, as a vacuum is applied to the skin surface directly over the axis defined by the needle 58, the fascia 16 separates from the skin, i.e., from the dermis 14 and epidermis 12. This separation is facilitated by the vent hole 75 located in the needle hub 68, which assists in breaking the surface tension between the skin and the fascia. FIG. 6 is a graphical depiction of a cavity 94 created below the skin due to the combination vacuum applied to the skin surface 22 and electrical current supplied to the muscles subjacent the vacuum source. This cavity region is also known in the medical field as a dead space or a bloodless plane.

The cavity 94 forms almost instantaneously as the flow of current is applied to the needle. In one exemplary embodiment, a small volume of pressurized gas is sent to the needle assembly 34 to push medications into the cavity shortly following the flow of current. The pressurized gas can be a low pressure gas of about 1-3 psig and a flow volume of about 0.02 cc to about 0.8 cc, which would be equivalent to the volume of medications injected into the cavity 94 from the dispensing tube 70. In one exemplary embodiment, the controller automatically senses the vacuum inside the interior cavity 87 of the housing 36, supply a current to the needle 58 and dispensing tube 70 when an appropriate vacuum is sensed, and delivers a quantity of pressurized gas to the needle a short time interval following the supply of current to the dispensing hub 72 to then deliver medications to the cavity 94. Depending on the treatment, medications delivered to the cavity can be any number of products including fillers and neurotoxin. However, medications can be any number of medications depending on the type of treatment or preventative care in question.

With reference to FIG. 4 in addition to FIG. 6, if a different injection assembly is used, such as the injection assembly 80 of FIG. 4, then the injection process includes placing the injection module 32 over an area to be injected, creating a vacuum using a vacuum pump 82 or other vacuum source, such as a separate vacuum pump, opening the valve 84 connected to the needle assembly 84, and then sending a current from the controller 30 to the needle. A cavity will form as previously discussed. However, rather than supplying pressurized gas to inject medications to the cavity 94, medications are automatically drawn into the cavity 94 due to a vacuum that is formed as the cavity is created. The valve 84 may be closed shut following a brief moment, such as 2-6 seconds following the flow of current. A second injection can now be made by moving the injection module 32 to a different location to be injected and repeating the described steps. As the housing 36 is under a vacuum, the vacuum breaker 56 should be activated to release the vacuum.

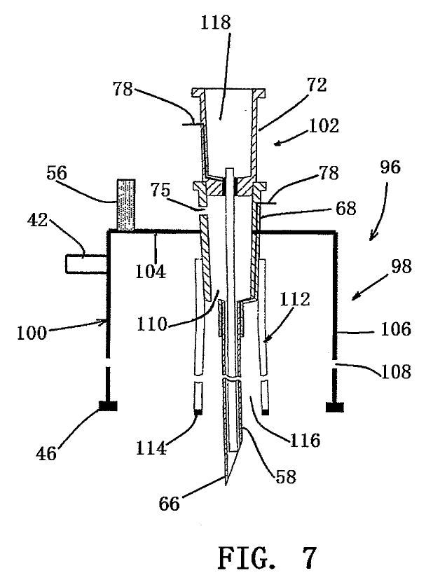

Referring now to FIG. 7, a semi-schematic cross-sectional side view of yet another alternative injection assembly 96 provided in accordance with aspects of the present invention is shown. The injection assembly comprises an injection module 98 and a master controller (not shown) similar to the controllers previously discussed. In one exemplary embodiment, the injection module 98 comprises a housing 100 comprising a nozzle 42, a vacuum breaker 56, and a needle assembly 102 attached to the housing. In one exemplary embodiment, the needle assembly 102 is attached to a top surface 104 of the housing 100, which may be an integrally formed top surface or a separate cap to be connected to the wall enclosure 106. The wall enclosure 106 is shown with a break line 108 representing a variable housing length to be determined depending on the needle and needle assembly. A pliable seat 46 is incorporated at the end edge of the wall enclosure 106 to serve as a soft seating surface.

In one exemplary embodiment, the needle assembly 102 is similar to the needle assembly shown in FIG. 3 with a few changes. In particular, a second opening 110 is incorporated in the needle hub 68 and an elongated shell 112 comprising a pliable seat 114 is coaxially disposed with the needle 58. The elongated shell 112 may be attached to the needle hub 68 by either interference fit or threaded engagement. An interior space 116 is defined interiorly of the shell 112, which is in communication with the first opening 75 and second opening 110 on the needle hub 68, which is in communication with the atmosphere.

When the injection assembly 96 is used on a patient, such as that shown in FIGS. 4 and 5, and the housing is under a vacuum, the space around the needle defined by the shell 112 is not in a vacuum whereas the space outside the shell 112 and inside the housing 100 is under a vacuum. Thus, when a current is applied to the leads 78 on the needle hub 68 and the dispensing hub 72, muscles will contract along the Z-axis and will tend to pull the skin located inside the shell 112 in the same direction. The skin, however, is held secured by the vacuum around the area between the shell 112 and the housing 100. This configuration, as compared to that shown in FIGS. 4 and 5, has been found to effectively stimulate a cavity near the needle tip 66 for depositing medicament stored inside the reservoir 118 defined by the dispensing hub 72. Similar to previously described embodiments, medicament may be dispensed using a pressurized gas source coupled to the reservoir 118 or may be gravity fed using a combination valve and tubing.

The interface between the needle hub 68 and the housing 100, and particularly the top surface 104 of the housing, may be any known prior art attachment means, including interference fit, friction fit, and threaded engagement. Preferably, the interface allows adjustment to the needle so that the needle tip extension, and therefore the depth of penetration of the needle, beyond the end edge of the housing 100 may be adjusted.

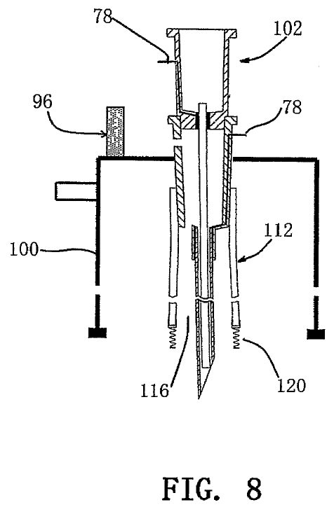

FIG. 8 is a semi-schematic cross-sectional view of the injection assembly 96 of FIG. 7 with an accordion seal 120 attached to the end edge of the elongated shell 112. The accordion shell 120 may be made from an elastomeric material and may be attached to the shell using detents or tongue and groove arrangement. The accordion seal 120 allows the interior space to remain constant by flexing and compensating for different curvatures of the face as the injection assembly 96 is moved from one injection site to another.

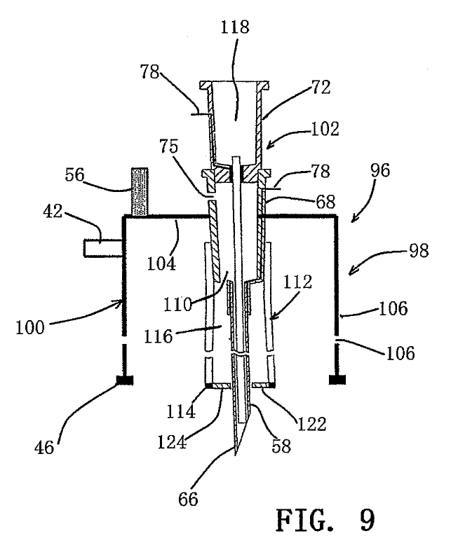

FIG. 9 is a semi-schematic cross-sectional side view of the injection assembly 96 of FIG. 7 with a leveling plate 122 attached to the opening of the elongated shell 112 and having an opening 124. In one exemplary embodiment, the leveling plate comprises a thermoplastic plate. The plate 122 is configured to provide a base line or an injection site that is level relative to the needle tip. Thus, when vacuum is applied to the skin for an injection, the area under the needle ensures that any skin that is raised by the vacuum is leveled by the plate 122. This in turn ensures that the injection site is level from site to site so that the depth of the injection is the same or nearly the same from site to site.

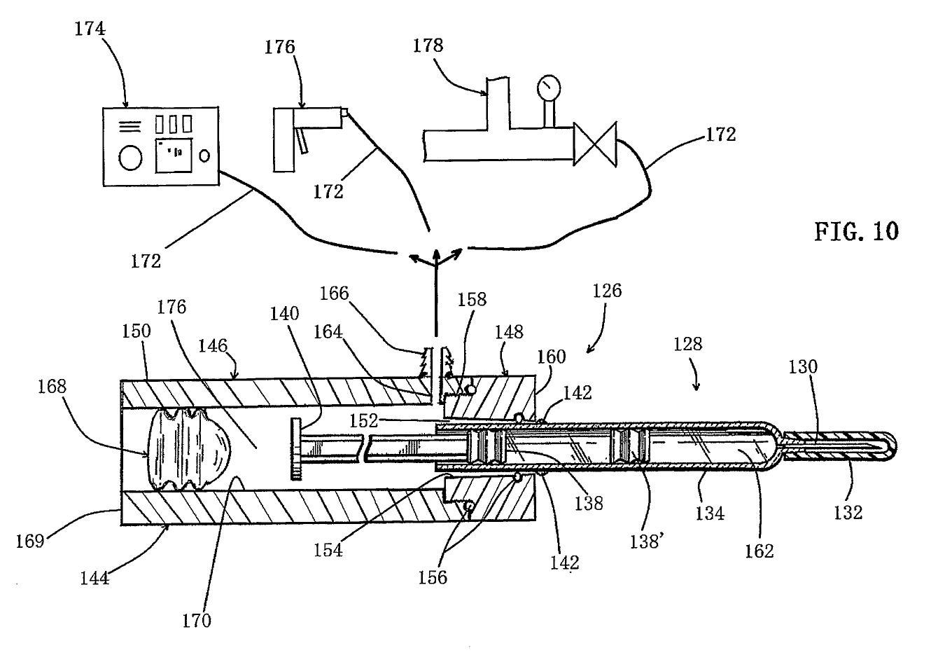

FIG. 10 is a semi-schematic side view of yet another injection assembly provided in accordance with aspects of the present invention, which is generally designated 126. In one exemplary embodiment, the injection assembly 126 comprises a syringe 128 comprising a needle 130, shown with a needle cap 132, a barrel 134, a plunger 136 having a push flange 140, and a piston 138. The syringe 128 may be any number of prior art syringe, which may include an integrated needle as shown or a separate needle with needle hub. In the figure, a second piston 138′ is shown distally advanced inside the barrel 134, which depicts an injection wherein the piston 138 is advanced from a proximal position on the barrel 134 to a more distal position. In one exemplary embodiment, the barrel 134 incorporates one or more stoppers 142 located on its external surface for registering the barrel relative to an injection housing, as further discussed below.

The injection assembly 126 further comprises an injection module 144 comprising a housing 146 comprising a loading cap 148 and an injection body section 150. The housing 146 may be made from a rigid thermoplastic material or a metal, such as aluminum or stainless steel. In one exemplary embodiment, the loading cap 148 comprises a bore 152 comprising a tapered cylindrical wall surface, tapers inwardly from a proximal point to a distal point, near a groove for accommodating an O-ring 156. In one exemplary embodiment, the cap 148 comprises a shoulder comprising a threaded end 158 comprising a second groove for accommodating a second O-ring 156. The cap 148 is configured to slide onto the barrel from the rear end of the barrel 134, where the plunger 136 projects through the barrel. The cap is pushed distally forward until the end edge 160 of the cap contacts the one or more stoppers 142.

Medication 162 may now be filled into the barrel by aspirating the plunger 136 to draw a vacuum. The injection body section 150 is now threaded to the cap 148 and the split-line between the body section 150 and the cap 148 sealed by a second O-ring 156. As shown, the injection body section 150 comprises a vent port or opening 164 comprising a nozzle 166, which may be a barb connector, and an activating piston 168 in dynamic sealing arrangement with the interior wall surface 170 of the body section 150.

The injection assembly 126 is configured to deliver an injection not by pushing the plunger 136 with a finger, such as a thumb, but by activating the activating piston 168 using a vacuum source. In one exemplary embodiment, the nozzle 166 on the body section 150 is connected to a vacuum source using a hose 172. The vacuum source can be any one of an electric vacuum pump 174, a manual hand vacuum pump 176, or a vacuum header 178, typically in hospitals or other institutions.

When the interior cavity 176 of the injection module 144 is subjected to a vacuum, the activating plunger 168 is automatically drawn distally. At some point, the activating plunger 168 will contact the push flange 140 and pushes the push flange and the plunger 136 into the barrel 134, which in turn, via the piston 138, pushes medications 162 inside the barrel out of the needle 132. It has been found that a vacuum of as little as 9-14 psia will effectively move the activating plunger 168 to then push the plunger 136. However, for injecting a more viscous fluid, a vacuum of about 1-6 psia may be required.

In an alternative embodiment, the piston 168 is fixed to the housing 146, i.e., does not move relative to the housing. The piston 168 can thus be an end cap or the like that can either be permanently secured to the open end 169 of the housing 146 or removable from the open end 169, such as by incorporating threads. In the present alternative embodiment, the barrel 134 of the syringe is configured to move into the housing 146 upon exposing the nozzle 166 to a vacuum source. In one exemplary embodiment, the stoppers 142 on the exterior surface of the barrel 134 are eliminated. Hence, when the interior cavity 176 is under a vacuum the barrel 134 and the plunger 136 both move proximally into the housing 146. At some point, the push flange 140 on the plunger hits the stationary piston 168 while the barrel 134 continues to move. This motion causes the plunger 138 to eventually contact and push medications inside the barrel out of the needle 130. In short, the present alternative embodiment is configured to discharge fluid out of the needle 130 while at the same time move the barrel 134 proximally relative to the injection module.

Although limited embodiments of the injection device and methods of using same have been specifically described and illustrated herein, many modifications and variations will be apparent to those skilled in the art. For example, separate controllers for controlling different functions may be incorporated instead of just one, different ways to supply current to the facial tissue using different means instead of via the leads in the needle and catheter hubs as described, and different ways to stimulate a void or cavity instead of using a stimulator in combination with a vacuum. Furthermore, it is understood and contemplated that features specifically discussed for one injection assembly may be adopted for inclusion with another injection assembly, provided the functions are compatible. Accordingly, it is to be understood that the injection assemblies and their components constructed according to principles of this invention may be embodied other than as specifically described herein. The invention is also defined in the following claims.

What is claimed is:

1. An injection apparatus comprising:
a needle assembly comprising a needle mounted to a housing;
a vacuum source connected to the housing for creating a vacuum inside the housing; and
a power supply directly or indirectly connected to the needle for supplying a current to the needle.

2. The injection apparatus of claim 1, further comprising a vacuum breaker.

3. The injection apparatus of claim 1, further comprising a nozzle on the housing.

4. The injection apparatus of claim 1, wherein the needle assembly comprising a needle hub having an opening exposed to atmosphere.

5. The injection apparatus of claim 1, further comprising an elongated shell coaxially disposed over the needle.

6. The injection apparatus of claim 5, wherein the elongated shell comprises a leveling plate.

7. The injection apparatus of claim 1, wherein the needle assembly comprises a metal lead.

8. The injection apparatus of claim 1, further comprising a delivery tube disposed annularly of the needle.

9. A method for injecting a subject comprising:
(a) placing an injection apparatus on a skin of the subject; the injection apparatus comprising:
(1) a needle assembly comprising a needle mounted to a housing;
(2) a vacuum source connected to the housing;
(3) a power supply directly or indirectly connected to the needle for supplying a current to the needle;
(b) penetrating the skin of the subject with the needle; and
(c) delivering an injection to the subject.

10. The method for injecting as recited in claim 9, further comprising the step of creating a cavity below the dermis of a skin.

11. The method for injecting as recited in claim 10, further comprising delivering the injection in the cavity.

12. The method for injecting as recited in claim 10, an opening in a needle hub of the needle assembly, the opening being exposed to atmospheric pressure.

13. A method for injecting a subject comprising:
penetrating a skin with a needle;
stimulating a cavity below the skin using an electric stimulator and a vacuum source; and
delivering medication to the cavity.

14. The method for injecting as recited in claim 13, further comprising the step of connecting an injection module to the vacuum source.

15. An injection apparatus comprising an injection module housing defining an interior cavity comprising a nozzle for connecting to a vacuum source, a first receiving end for receiving a syringe, and a second receiving end for accommodating an end object for sealing the second receiving end, wherein the nozzle is configured to communicate with a vacuum source to place the interior cavity under a vacuum.

16. The injection apparatus of claim 15, further comprising a syringe mounted at the first receiving end of the housing.

17. The injection apparatus of claim 16, wherein the end object is a movable piston.

18. The injection apparatus of claim 15, wherein the injection module housing comprises a split line for separating the housing into at least two housing components.

19. The injection apparatus of claim 15, further comprising a groove for accommodating an O-ring.

20. The injection apparatus of claim 15, wherein the end object is a movable piston.

21. The injection apparatus of claim 16, wherein a barrel of the syringe is configured to move inside the interior cavity of the injection module housing.
